(19) United States
(12) Patent Application Publication
(10) Pub. No.: US 2017/0049966 A1
(43) Pub. Date: Feb. 23, 2017

(54) COAXIAL PISTON INJECTOR

(71) Applicant: Auckland UniServices Ltd., Auckland (NZ)

(72) Inventors: Andrew James Taberner, Auckland (NZ); Bryan Paul Ruddy, Auckland (NZ); Rhys Matthew James Williams, Auckland (NZ); James William McKeage, Auckland (NZ); Poul Michael Fonss Nielsen, Auckland (NZ)

(21) Appl. No.: 15/240,400

(22) Filed: Aug. 18, 2016

Related U.S. Application Data
(60) Provisional application No. 62/206,943, filed on Aug. 19, 2015.

(51) Int. Cl. A61M 5/30; A61M 5/315

(52) U.S. Cl. CPC A61M 5/3007; A61M 5/31515; A61M 5/31505

(57) ABSTRACT

An apparatus for delivering an injectate to a patient includes a piston mechanism including an outer piston. The outer piston is slideably disposed in a chamber of the apparatus and has a channel extending therethrough. The piston mechanism also includes an inner piston including a stop and a plunger element, the plunger element being slideably disposed in the channel of the outer piston. The piston mechanism is configurable into a number of configurations including a first configuration in which the stop of the inner piston is not in contact with the outer piston such that the inner piston is movable in the channel without causing movement of the outer piston and a second configuration in which the stop of the inner piston is in contact with the outer piston such that the inner piston and the outer piston move together in the chamber.

### CROSS-REFERENCE TO RELATED APPLICATIONS

This application claims the benefit of U.S. Provisional Application No. 62/206,943 filed Aug. 19, 2015.

### BACKGROUND

The skin of organisms such as humans serves as a protective barrier that, among other functions, prevents pathogens from entering the body and prevents or regulates fluids such as blood and water from exiting the body. In the field of modern medicine, there is often a need to deliver injectates such as drugs through the skin and into the bloodstream or tissue of patients. Traditionally, this delivery of liquids into a patient's body is accomplished by having a technician insert a needle through the patient's skin and into an area inside of the patient's body where the liquid can enter the patient's blood stream.

However, the use of needles to deliver liquids into a patient's body has a number of significant drawbacks such as the pain associated with being pierced by a needle, the fear that many patients have of needles, and the skin damage and associated risk of infection that occurs due to the use of needles.

As a result, needle-free transdermal injection devices have been developed. These devices use a high pressure, narrow jet of injectate (e.g., injection liquid or powder) to penetrate a patient's skin, obviating the need to pierce the patient's skin with a needle.

### SUMMARY

Aspects relate to the use of a series of nested pistons articulated by a single force-producing mechanism in order to create a time-varying pressure source matched to requirements of needle-free jet injection.

In a general aspect, an apparatus for delivering an injectate to a patient includes a chamber for holding the injectate, the chamber having an orifice through which the injectate is delivered disposed at a chamber distal end. A piston mechanism is disposed at least partially in the chamber and includes an outer piston slideably disposed in the chamber. The outer piston has a channel extending through the outer piston from a proximal end of the outer piston to a distal end of the outer piston. The piston mechanism also includes an inner piston having an inner piston proximal end including a stop and an inner piston distal end including a plunger element. The plunger element is slideably disposed in the channel of the outer piston. The apparatus also includes a driver element coupled to the inner piston and configured to cause movement of the piston mechanism in the chamber for delivery of the injectate. The piston mechanism is configurable into a number of configurations including a first configuration in which the stop of the inner piston is not in contact with the outer piston such that the inner piston is movable in the channel without causing movement of the outer piston and a second configuration in which the stop of the inner piston is in contact with the outer piston such that the inner piston and the outer piston move together in the chamber.

Aspects may include one or more of the following features.

The apparatus may include one or more stop elements disposed at the chamber distal end for preventing withdrawal of outer piston from the chamber. The channel of the outer piston may include a shoulder configured to interact with the plunger element of the inner piston to prevent withdrawal of the inner piston from the channel. The channel may include a restricted portion with a first channel inner diameter and an expanded portion with a second channel inner diameter, the first channel inner diameter being less than the second channel inner diameter. The shoulder may be formed at a point of transition between the restricted portion and the expanded portion.

An outer diameter of the plunger element may be substantially the same as the second channel inner diameter. An outer diameter of the plunger element at the proximal end of the inner piston and an outer diameter of the stop at the distal end of the inner piston may both be larger than the first channel inner diameter. A diameter of the orifice may be in a range of 0.1 mm to 0.5 mm. A diameter of the plunger element may be in a range of 2 mm to 7 mm. A diameter of the outer piston may be in a range of 2.5 mm to 36 mm. A ratio of an outer diameter of the outer piston to a diameter of the plunger may be in a range of 1.25 to 6.

An outer diameter of the outer piston may be substantially the same as an inner diameter of the chamber. The apparatus may include an injection controller for causing the driver element to apply a substantially constant force to the inner piston for a duration of an injection operation. The force may be in a range of 30 N to 1000 N. The apparatus may be configured to deliver a total volume of injectate in a range of 0.1 mL to 15 mL to the patient. The apparatus may be configured such that 5% to 20% of the total volume of injectate is delivered to the patient when the piston mechanism is in the first configuration and 80% to 90% of the total volume of injectate is delivered to the patient when the piston mechanism is in the second configuration. The apparatus may be configured to deliver a volume of injectate to a depth in a range of 5 mm to 25 mm under the patient's skin.

In general, needle-free injection devices are mechanical devices which apply force to a piston disposed in a chamber filled with injectate to produce a high-velocity liquid jet of injectate from the injection device. The force applied to the piston is typically generated using springs or compressed gas and results in a substantially constant jet velocity profile over the course of an injection due to the force development of the injectors being substantially constant or gradually decreasing over the course of an injection. Some needle-free injection devices use an interchangeable or manually adjustable piston to set an injection volume.

In some examples it is desirable to deliver the narrow jet of injectate to the patient's skin in two phases. In a first phase, the velocity of the injectate is sufficient to pierce a small hole in the patient's skin and to penetrate to a desired delivery depth in the patient's tissue. In a second phase, when the desired delivery depth has been reached, the velocity of the injectate is reduced such that the injectate penetrates little further into the patient's tissue but is sufficient to continue diffusing injectate into the surrounding tissues, resulting in the desired volume of injectate being delivered. Typically, the volume of drug required to initially penetrate the tissue is a relatively small percentage of the total volume to be delivered.

Aspects described herein accomplish two-phase injection of injectate by using a simple, mechanical injector mechanism to transform a substantially constant force input into a time-varying pressure in the injectate in the injection chamber of an injection device. The time-varying pressure results in a time-varying velocity in the jet of injectate expelled from the needle-free injection device.

In particular, the injector mechanism initially operates in a first configuration, which produces a short duration ‘piercing’ jet, with a velocity sufficient to reach the desired injection depth during the process of injection-hole erosion. The injector mechanism then transitions into a second configuration, which produces a lower velocity, ‘delivery’ jet, sufficient to deliver the drug through the existing injection hole without further eroding the injection-hole. The dual configuration of the injector mechanism effectively delivers injectate through a single nozzle in a smooth, precisely controlled two-phase injection sequence with a brief initial high-velocity phase and a longer low-velocity phase that delivers the balance of the fluid to be injected.

Aspects may include one or more of the following advantages.

Among other advantages, aspects efficiently achieve a two-stage injection process without requiring an electric motor that can both produce high force during the “piercing” phase of delivery and a relatively low force during the “delivery phase.”

Aspects exploit the smooth, precise operation of a single motor for large volume injections by better matching the force-production performance of the motor to the injection requirements throughout both phases of a two-phase injection process.

Aspects require only a single actuator and thus can be built with minimal increase in cost or control complexity.

Aspects include a single fluid reservoir which is in communication with two or more pistons at all times without requiring the use of valves.

The injection device is fully reversible for reloading the injectate chamber with injectate, by applying a retraction force via the same force source that performs the injection.

Other features and advantages of the invention are apparent from the following description, and from the claims.

### DESCRIPTION OF DRAWINGS

FIG. 1 is a needle-free transdermal injection device.

FIG. 2 is a diagram of an internal configuration of the needle-free transdermal injection device of FIG. 1.

FIG. 3 is the needle-free transdermal injection device of FIG. 2 operating in a piercing phase.

FIG. 4 is the needle-free transdermal injection device of FIG. 2 transitioning from the piercing phase to a delivery phase.

FIG. 5 is the needle-free transdermal injection device of FIG. 2 operating in the delivery phase.

FIG. 6 is the needle-free transdermal injection device of FIG. 2 upon completion of the delivery phase.

FIG. 7 is a graph of injection velocity over time.

FIG. 8 is a graph of injection depth over time.

### DESCRIPTION

### 1 Needle-Free Transdermal Injection Device

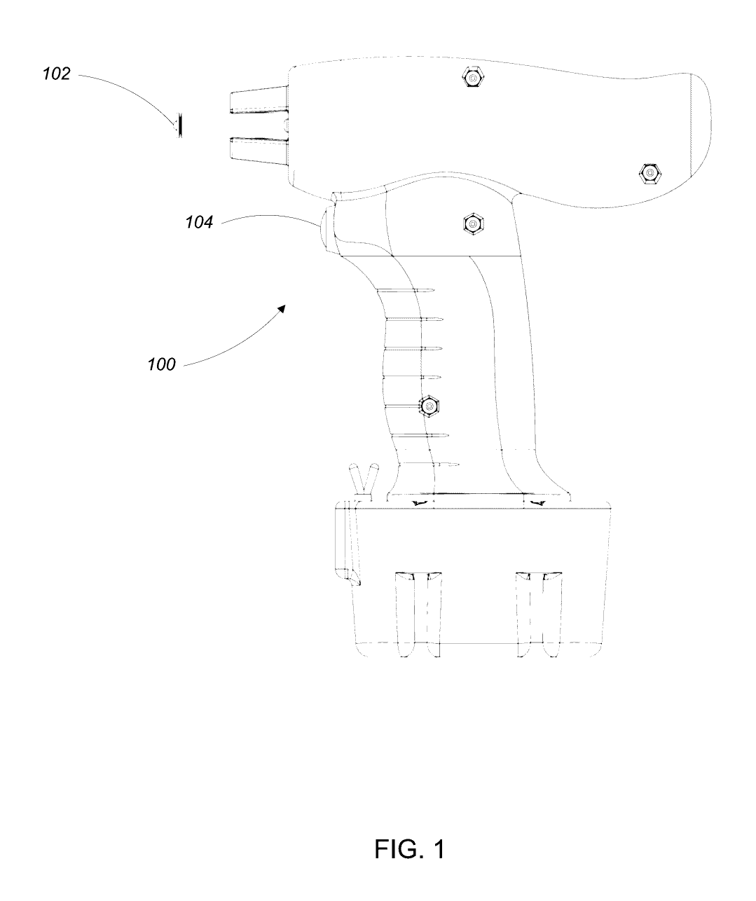

Referring to FIG. 1, a needle-free transdermal injection device 100 includes an injector mechanism (not shown), including an injectate, disposed therein. Very generally, upon actuation of a pushbutton 104, the injection device 100 operates the injector mechanism through a two-phase injection process to eject the injectate from a chamber within the injector mechanism and out of an injection nozzle 102 of the injection device 100. The ejected injectate is delivered through the skin and into the bloodstream of a patient.

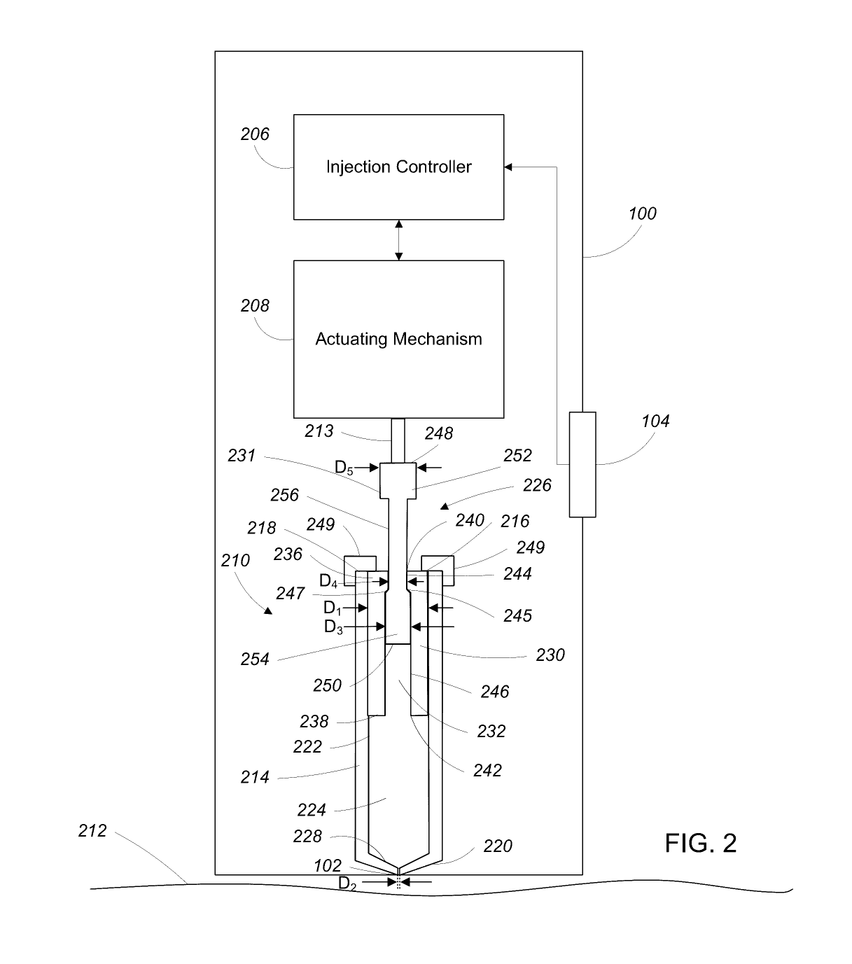

Referring to FIG. 2, the needle-free transdermal injection device 100 is positioned with the injection nozzle 102 adjacent to a patient's skin 212 in preparation for delivering injectate through the patient's skin 212. The injection device 100 includes an injection controller 206, an actuating mechanism 208, and the injector mechanism 210.

In some examples, the actuating mechanism 208 includes an electromagnetic linear motor (e.g., a Lorentz-force linear actuator of the type described in U.S. Pat. No. 7,833,189; a linear actuator driven by a rotary motor through a ball drive mechanism, a linear actuator driven by a rotary motor through a rack-and-pinion mechanism, etc.). A linkage 213 couples the actuator mechanism 208 to the injector mechanism 210. In operation, the injection controller 206 servo-controls the actuating mechanism 208 such that a substantially constant force applied to the injector mechanism 210 via the linkage 213.

### 1.1 Injector Mechanism

The injector mechanism 210 includes a body 214 with a body proximal end 218 and a body distal end 220. A piston receiving opening 216 having a first diameter, D₁ is disposed at the body proximal end 218. The injection nozzle 102 is disposed at the body distal end 220 and has a second diameter, D₂. An injectate chamber 222 is formed in and extends along a length of the body 214 from the piston receiving opening 216 to the injection nozzle 102 and is configured to receive injectate 224. Except for a tapered chamber portion 228 near the body distal end 220, the injectate chamber 222 has substantially the same diameter, D₁ as the piston receiving opening 216.

### 1.1.1 Nested Piston

A nested piston mechanism 226 extends through the piston receiving opening 216 and is at least partially disposed in the injectate chamber 222. The nested piston mechanism 226 includes an outer piston 230 and an inner piston 231.

### 1.1.1.1 Outer Piston

The outer piston 230 is disposed in the injectate chamber 222 and has an outer piston proximal end 236 and an outer piston distal end 238. An outer diameter of the outer piston 230 is substantially the same as the diameter, D₁ of the piston receiving opening 216 and the injectate chamber 222 such that a substantially liquid-tight seal is established at a contact surface between the outer piston 230 and an inner wall of the injectate chamber 222.

A channel 232 extends through the outer piston 230 from a first outer piston opening 240 disposed at the outer piston proximal end 236 to a second outer piston opening 242 disposed at the outer piston distal end 238.

In some examples, over the length of the channel 232, the channel transitions from an expanded portion 246 to a restricted portion 244 at a transition point 245. The expanded portion 246 extends between the transition point 245 and the second outer piston opening 242 and has an inner diameter, D₃. The restricted portion 244 extends between the first outer piston opening 240 and the transition point 245 and has an inner diameter, D₄ which is less than the inner diameter, D₃ of the restricted portion 244 of the channel 232. A shoulder 247 is formed at the transition point 245.

The body proximal end 218 includes one or more stop members 249 that prevent movement of the outer piston 230 out of the injectate chamber 222. In general, by preventing movement of the outer piston 230 out of the injectate chamber 222 during an injection, injectate is prevented from redistributing into a volume created by movement of the outer piston 230 out of the chamber 222. Injectate is thus forced out of the injection nozzle 102.

### 1.1.1.2 Inner Piston

The inner piston 231 has an inner piston proximal end 248 and an inner piston distal end 250. The inner piston 231 includes a back stop 252 disposed at the inner piston proximal end 248, a plunger 254 disposed at the piston distal end 250, and a shaft 256 extending between the back stop 252 and the plunger 254.

The plunger 254 is slidably disposed in the expanded portion 246 of the channel 232 and has an outer diameter substantially matching the inner diameter, D₃ of the expanded portion 246 of the channel 232 such that a liquid-tight seal is established at a contact surface between the plunger 254 and the expanded portion 246 of the channel 232. In some examples, since the diameter of the plunger 254, D₃ is greater than the inner diameter, D₄ of the restricted portion 244 of the channel 232, withdrawal of the plunger 254 from the channel 232 through the first outer piston opening 240 is prevented by the restricted portion 244 of the channel 232 (i.e., the plunger 254 is unable to move past the shoulder 247).

The shaft 256 of the inner piston 231 extends from the plunger 254, through the restricted portion 244 of the channel 232, and out of the proximal end 218 of the outer piston 230 via the first outer piston opening 240.

The back stop 252 of the inner piston 231 is disposed outside of the channel 232 and is coupled to the actuating mechanism 108 via the linkage 213. In some examples, an outer diameter, D₅ of the back stop 252 is greater than the inner diameter, D₄ of the restricted portion 244 of the channel 232, thereby restricting movement of the inner piston 231 by preventing the back stop 252 of the inner piston 231 from moving past the outer piston proximal end 236 and into the channel 232.

In some examples, the configuration shown in FIG. 2 is especially useful when the injector mechanism 210 is designed as a reusable injector mechanism since the restricted portion 244 of the channel 232 forms a shoulder 247 that is used to reset the injector mechanism 210. For example, after a desired volume of injectate is delivered to a patient, the motor slows to a halt and then reverses. When the motor reverses, the inner piston 231 first retracts until the proximal end of plunger 254 contacts shoulder 247, and then the outer piston retracts with the inner piston 231 until both pistons are arranged in their original positions. As the pistons retract, a negative pressure is established in the injectate chamber causing fresh drug to be drawn into the injectate chamber through a check-valve (not shown), preparing the device for the next delivery. In some examples, the injectate chamber is re-filled manually. However, as is described in greater detail below, certain configurations of the injector mechanism 210 are not intended for re-use and therefore may not require that a shoulder 247 is formed in the channel 232.

### 2 Operation

In the configuration shown in FIG. 2, the needle-free transdermal injection device 100 has its injectate chamber 222 filled with a desired amount of injectate 224 and is prepared to inject the injectate through the patient's skin 212 and into the tissue below.

### 2.1 Piercing Phase

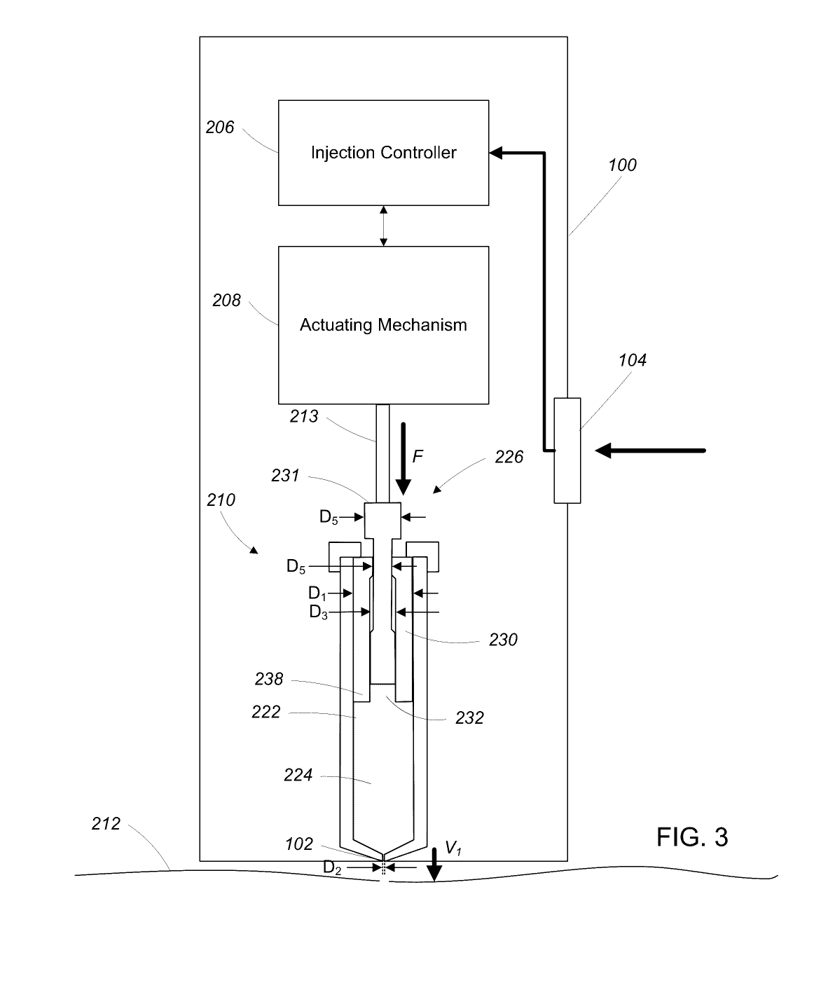

Referring to FIG. 3, when a user actuates the pushbutton 104, the injection controller 206 initiates a first, piercing phase in which a jet of injectate is ejected from the injection nozzle 102 with sufficient velocity to pierce the patient's skin 212.

To do so, the injection controller 206 causes the actuating mechanism 208 to apply a substantially constant force, F to the linkage 213. The force applied to the linkage 213 causes the inner piston 231 of the nested piston mechanism 226 to move through the channel 232 of the outer piston 230 in a direction toward the outer piston distal end 238. The movement of the inner piston 231 through the channel causes a first pressure, P₁ in the injectate 224 in the injectate chamber 222, which in turn causes ejection of the injectate 224 from the injection nozzle 102 at a first velocity, V₁. The magnitude of the first pressure, P₁ depends on the force, F applied to the linkage 213 and the area, A₃ of the plunger 254 (where A₃=π(D₃/2)²) as follows:

P₁=F/A₃+101,325 N/m²

The first velocity, V₁ depends on the applied force, F, the area, A₃ of the plunger 254, and the area, A₂ of the opening of the injection nozzle 102 (where A₂=π(D₂/2)²) as follows:

$${V}_{1} = \sqrt{\frac{2 ( \frac{F}{{A}_{3}} )}{ρ ( 1 - ( \frac{{A}_{2}}{{A}_{3}} ) )}}$$

where ρ is the density of the injectate 224.

Since the force, F applied to the linkage 213 is substantially constant, the injection velocity, V₁ is substantially constant as the inner piston 231 travels through the channel 232.

### 2.2 Delivery Phase

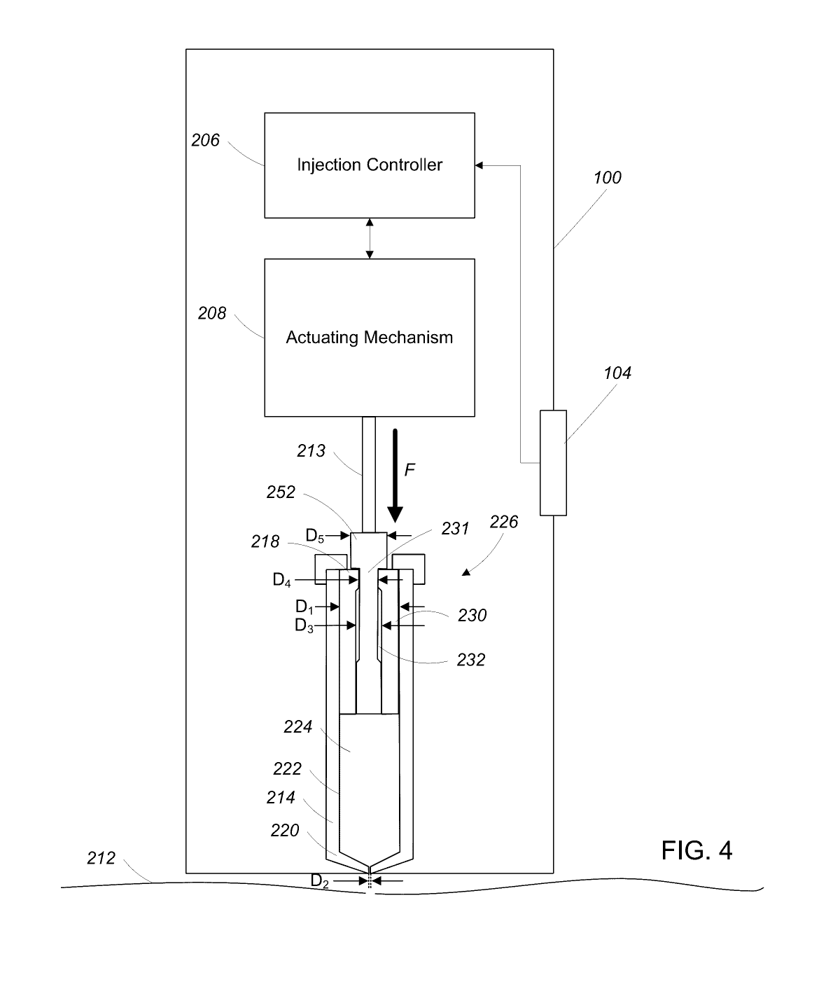

Referring to FIG. 4, a second, delivery phase is initiated, in which the force, F applied to the linkage 213 by the actuator mechanism 208 moves the entire nested piston mechanism 226 through the injectate chamber 222. The substantially larger piston area of the nested piston mechanism 226 generates a correspondingly lower pressure in the injectate, resulting in an injection velocity suitable for the delivery of the bulk of the injectate 224 without substantially increasing the injection depth.

The delivery phase begins when the back stop 252 of the inner piston 231 makes contact with the outer piston proximal end 218. Since the back stop 252 is unable to travel into the channel 232 of the outer piston 230, the back stop 252 transfers force to the outer piston 230, causing both the outer piston 230 and the inner piston 231 (i.e., the entire nested piston mechanism 226) to move along the injectate chamber 222 in a direction toward the distal end 220 of the body 214. In some examples, the transition from the piercing phase to the delivery phase is managed electronically (e.g., by slowing the motor before the point of impact) and/or mechanically (e.g., through the use of a damped coupling).

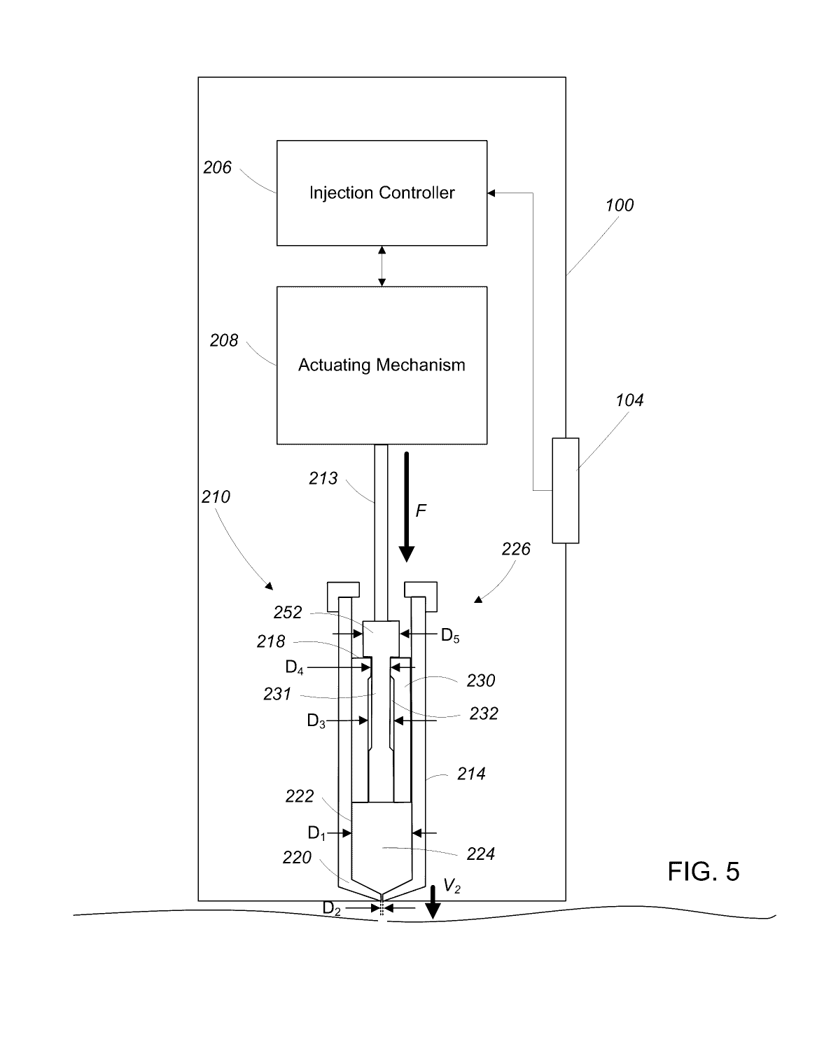

Referring to FIG. 5, The movement of nested piston mechanism 226 through the channel causes a second pressure, P₂, less than the first pressure, P₁ in the injectate 224 in the injectate chamber 222. The second pressure, P₂ causes ejection of the injectate 224 from the injection nozzle 102 at a second velocity, V₂, less than the first velocity, V₁. The magnitude of the second pressure, P₂ depends on and the force, F applied to the linkage 213 and the area, A₁ of the nested piston mechanism 226 (where A₁=π(D₁/2)²) as follows:

P₂=F/A₁+101,325 N/m²

The first velocity, V₂ depends on the applied force, F, the area, A₁ of the nested piston mechanism 226, and the area, A₂ of the opening of the injection nozzle 102 as follows:

$${V}_{2} = \sqrt{\frac{2 ( \frac{F}{{A}_{1}} )}{ρ ( 1 - ( \frac{{A}_{2}}{{A}_{1}} ) )}}$$

where ρ is the density of the injectate 224.

Since the force, F applied to the linkage 213 is substantially constant, the injection velocity, V₂ is substantially constant as the nested piston mechanism 226 travels through the channel 232.

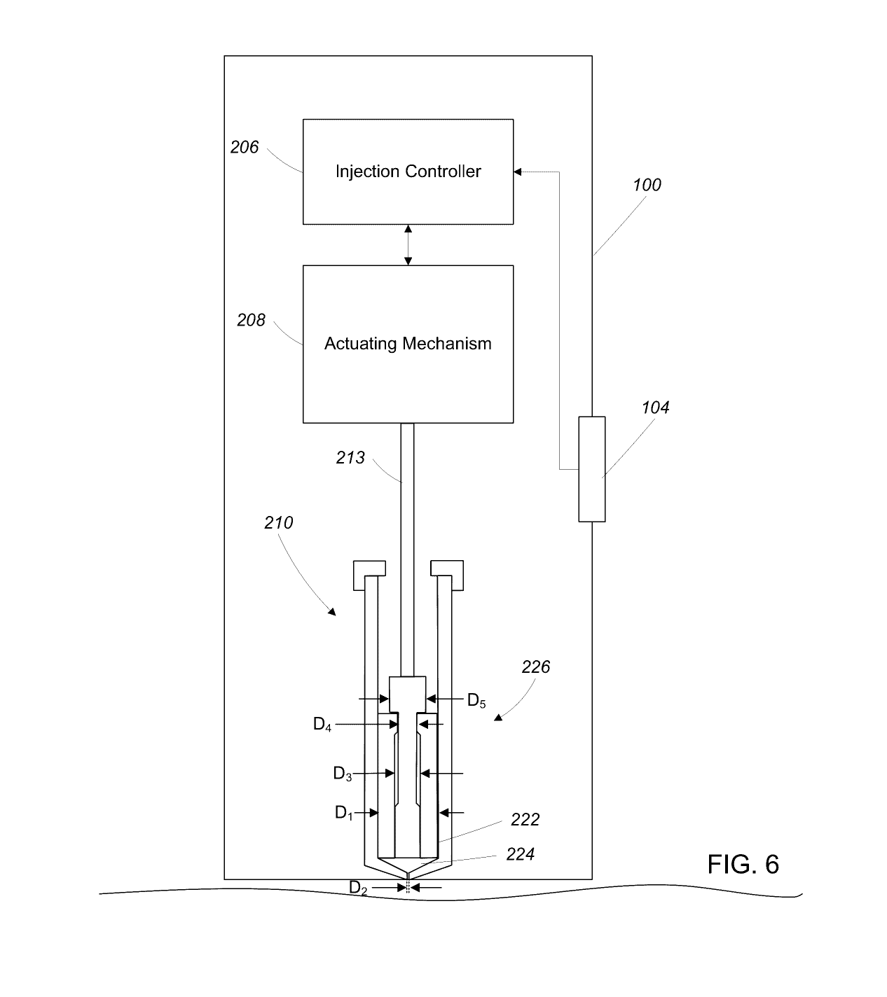

Referring to FIG. 6, the nested piston mechanism 226 either reaches the end of the injectate chamber 222 and/or the injection controller causes the actuating mechanism 208 to stop applying force to the linkage 213, wherein the ejection of injectate 224 from the injectate chamber 222 ceases and the injection is complete.

### 3 Exemplary Configuration

In one exemplary configuration of the injection device 210, the diameter, D₃ of the expanded portion 246 of the channel 232 is 3.56 mm, the diameter, D₁ of the injectate chamber 222 is 8.34 mm, and the diameter, D₂ of the injection nozzle 102 is 200 μm.

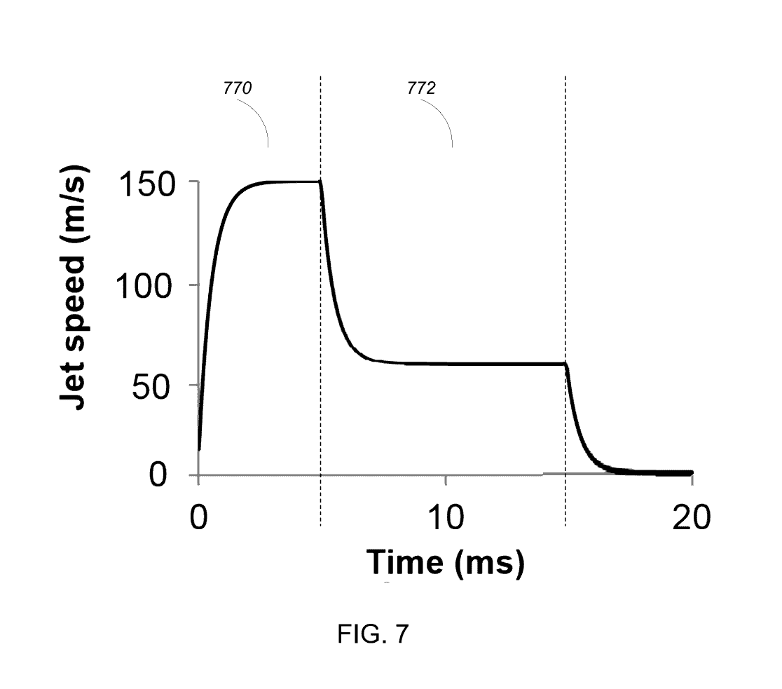

Referring to FIG. 7, with the above configuration, given a constant force, F of 110 N and an injectate density, ρ of 1000 kg/m³ (i.e., the density of a water injectate), the pressure, P₁ of the injectate 224 in the injectate chamber 222 is approximately 11 MPa during the first, piercing phase 770. The pressure, P₁ results in the injectate ejected from the nozzle 102 being ejected with a velocity, V₁ of approximately 150 m/s.

In the second, delivery phase 772, the pressure, P₂ of the injectate 224 in the injectate chamber 222 is approximately 2 MPa. The pressure, P₂ results in the injectate ejected from the nozzle 102 being ejected with a velocity, V₂ of approximately 65 m/s.

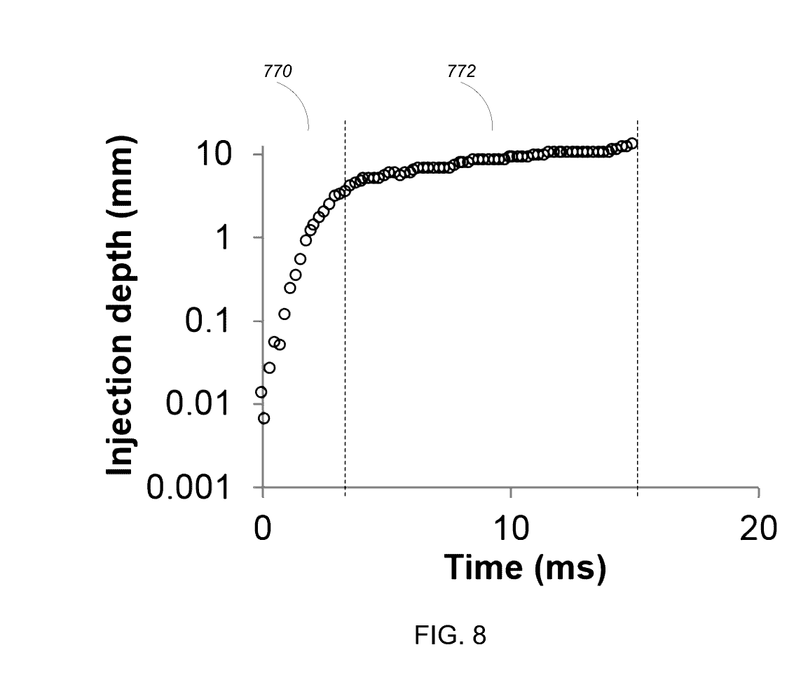

Referring to FIG. 8, a graph of injection depth over time illustrates a rapidly increasing injection depth during the piercing phase 770 and a slowly increasing injection depth during the delivery phase 772.

It is noted that the pressures and velocities described above are ideal and it is likely that the actual pressures and velocities will differ from the ideal values mentioned above. In particular, physical attributes such as friction, expansion of the body 214, injectate density, and barometric pressure, among other attributes will affect the actual pressures and velocities generated.

### 4 Alternatives

In some examples, the injector mechanism 210 is configured as a disposable, single-use injector mechanism. In such configurations, the channel 232 may have a single inner diameter along its length matching the outer diameter, D₃ of the plunger 254 (rather than restricted portion and an expanded portion with differing diameters). Due to the lack of a shoulder formed between a restricted portion and an expanded portion, retraction of the entire nested piston mechanism 226 by retraction of the inner piston 231 can not occur, thereby resulting in a single use configuration of the injector mechanism. In general, in the single-use injector mechanism configuration, the shaft of the inner piston 231 has an outer diameter that is less than or equal to the diameter, D₃ of the plunger 254.

While this embodiment benefits from the use of a controllable actuator for drug delivery, it can also be used to shape the pressure and jet velocity profile of a system actuated by other means, such as springs or compressed gases. This would significantly reduce the amount of mechanical work that must be done by such actuators to perform an injection, allowing the resulting injection system to be smaller, lighter, and/or more easily recharged.

In some examples, the total injectate volume is 1.2 mL and the volume of injectate used during the piercing phase is approximately 100 μL. In some examples, the piercing depth is in a range of 5 mm to 25 mm. In some examples, the injection occurs in a range of 5 ms to 25 ms. In some examples,

In some examples, a diameter of the orifice is in a range of 0.1 mm to 0.5 mm. In some examples, a diameter of the plunger element is in a range of 2 mm to 7 mm. In some examples, a diameter of the outer piston is in a range of 2.5 mm to 36 mm. In some examples, a ratio of an outer diameter of the outer piston to a diameter of the plunger is in a range of 1.25 to 6. In some examples, a for applied to the linkage is in a range of 30 N to 1000 N. In some examples, the apparatus is configured to deliver a total volume of injectate in a range of 0.1 mL to 15 mL to the patient. In some examples, 5% to 20% of the total volume of injectate is delivered to the patient when the piston mechanism is in the first configuration and 80% to 90% of the total volume of injectate is delivered to the patient when the piston mechanism is in the second configuration.

In some examples, force generation mechanisms other than motors are used to generate the force that propels the nested piston. For example, certain aspects are spring- or gas-driven system.

It is to be understood that the foregoing description is intended to illustrate and not to limit the scope of the invention, which is defined by the scope of the appended claims. Other embodiments are within the scope of the following claims.

What is claimed is:

1. An apparatus for delivering an injectate to a patient, said apparatus comprising:
a chamber for holding the injectate, the chamber having an orifice through which the injectate is delivered disposed at a chamber distal end;
a piston mechanism disposed at least partially in the chamber, the piston mechanism including
an outer piston slideably disposed in the chamber, said outer piston having a channel extending through the outer piston from a proximal end of the outer piston to a distal end of the outer piston, and
an inner piston having an inner piston proximal end including a stop and an inner piston distal end including a plunger element, the plunger element being slideably disposed in the channel of the outer piston; and
a driver element coupled to the inner piston and configured to cause movement of the piston mechanism in the chamber for delivery of the injectate;
wherein the piston mechanism is configurable into a plurality of configurations including
a first configuration in which the stop of the inner piston is not in contact with the outer piston such that the inner piston is movable in the channel without causing movement of the outer piston, and
a second configuration in which the stop of the inner piston is in contact with the outer piston such that the inner piston and the outer piston move together in the chamber.

2. The apparatus of claim 1 further comprising one or more stop elements disposed at the chamber distal end for preventing withdrawal of outer piston from chamber.

3. The apparatus of claim 1 wherein the channel of the outer piston includes a shoulder configured to interact with the plunger element of the inner piston to prevent withdrawal of the inner piston from the channel.

4. The apparatus of claim 3 wherein the channel includes a restricted portion with a first channel inner diameter and an expanded portion with a second channel inner diameter, the first channel inner diameter being less than the second channel inner diameter.

5. The apparatus of claim 4 wherein the shoulder is formed at a point of transition between the restricted portion and the expanded portion.

6. The apparatus of claim 4 wherein an outer diameter of the plunger element is substantially the same as the second channel inner diameter.

7. The apparatus of claim 4 wherein an outer diameter of the plunger element at the proximal end of the inner piston and an outer diameter of the stop at the distal end of the inner piston are both larger than the first channel inner diameter.

8. The apparatus of claim 1 wherein a diameter of the orifice is in a range of 0.1 mm to 0.5 mm.

9. The apparatus of claim 1 wherein a diameter of the plunger element is in a range of 2 mm to 7 mm.

10. The apparatus of claim 1 wherein a diameter of the outer piston is in a range of 2.5 mm to 36 mm.

11. The apparatus of claim 1 wherein a ratio of an outer diameter of the outer piston to a diameter of the plunger is in a range of 1.25 to 6.

12. The apparatus of claim 1 wherein an outer diameter of the outer piston is substantially the same as an inner diameter of the chamber.

13. The apparatus of claim 1 further comprising an injection controller for causing the driver element to apply a substantially constant force to the inner piston for a duration of an injection operation.

14. The apparatus of claim 13 wherein the force is in a range of 30 N to 1000 N.

15. The apparatus of claim 1 wherein the apparatus is configured for delivering a total volume of injectate in a range of 0.1 mL to 15 mL to the patient.

16. The apparatus of claim 15 wherein 5% to 20% of the total volume of injectate is delivered to the patient when the piston mechanism is in the first configuration and 80% to 90% of the total volume of injectate is delivered to the patient when the piston mechanism is in the second configuration.

17. The apparatus of claim 1 wherein the apparatus is configured to deliver a volume of injectate to a depth in a range of 5 mm to 25 mm under the patient's skin.
